(19) United States
(12) Patent Application Publication
(10) Pub. No.: US 2006/0029620 A1
(43) Pub. Date: Feb. 9, 2006

(54) Mycobacterium smegmatis vaccine

(76) Inventors: Guozhi Wang, Beijing (CN); Miao Xu, Beijing (CN); Baowen Chen, Beijing (CN); Xiaobing Shen, Beijing (CN); Cheng Su, Beijing (CN); Maoxiang Wang, Changchun (CN); Yuhe Yin, Changchun (CN); Yujie Ma, Chang Chun (CN)

Correspondence Address: DUANE MORRIS, LLP;IP DEPARTMENT, 30 SOUTH 17TH STREET, PHILADELPHIA, PA, 19103-4196, US

(21) Appl. No.: 10/913,626

(22) Filed: Aug. 5, 2004

(51) Int. Cl. A61K 39/04

(52) U.S. Cl. 424/248.1

(57) ABSTRACT

The present invention is about a mycobacterium smegmatis vaccine, a split product of mycobacterium smegmatis. This vaccine has made good use of the adjuvant function of bacterial cellwall and antigenic specificity of tropina which can stimulate the production of cytokine and consists of many immunologically competent CpG fragments of bacteria DNA. Such vaccine is prepared by treating newly-cultured thalli with a physical quassation technique. This vaccine is capable of enhancing normal immune function against TB, promoting the recovery of normal immune function in TB patients with hypofunction, and depressing the immunological responses in TB patients with hyperfunction. It is especially effective in the prevention and treatment of MTB carriers.

### TECHNICAL FIELD

The present invention makes public a biological product capable of immunological regulation, the Mycobacterium smegmatis vaccine, made from Mycobacterium smegmatis (CGMCC NO.0795). This technique is an application of microbiological theories.

### BACKGROUND OF THE INVENTION

TB is one of the most threatening diseases in the world. It is estimated that one-third of the world population, nearly 2,000,000,000 have been infected with MTB. Each year, nearly 10,000,000 new cases arise and 3,000,000 people die of it, which equals the total number of deaths from all other infectious diseases. For all those people infected with MTB and with a strong positive result in PPD tests(that is, the diameter of the induration no less than 15 mm, or bubbles and necrosis occurs), the incidence rate is higher; hence such populations are called TB high-risk people. Therefore, how to prevent the high-risk people from contracting TB has become a focus in global tuberculosis control.

At present, global tuberculosis control is realized through BCG vaccination among the normal population and DOTS (directly observing treatment short course) given to patients with clinical symptoms. However, BCG can only be vaccinated to the healthy people, and it cannot protect those who are MTB carriers. What is more, DOTS mainly depends on chemicals which is unacceptable to MTB carriers due to its long course of treatment and serious side effects. Therefore, the search for suitable prevention and treatment methods for those MTB carriers is of great significance to tuberculosis control. Furthermore, we believe that great importance should be attached not only to preventing pathogens from attacking hosts, but also to preventing carriers from being attacked by the disease.

M.S is a non-pathogenic fast-growing mycobacterium. The M.S vaccine, made from M.S, is of good immunological regulation and can be used as prevention and treatment for MTB carriers.

### CONTENT OF THE INVENTION

This invention is to provide an M.S vaccine, which is capable of enhancing normal immune function against TB, promoting the recovery of normal immune function in TB patients with hypofunction, and depressing the immunological responses in TB patients with hyperfunction. It is especially effective in the prevention and treatment of MTB carriers.

The other objective is to introduce the preparative method of the M.S vaccine as aqueous vaccines or lyophilized preparations.

The two objectives are fulfilled as follows.

The M.S vaccine is a split product of M.S CGMCC NO.0795. The optimizing M.S strains, labeled as CGMCC NO.0795, have been preserved by the China Committee for Culture Collection of Microorganisms since Sep. 10, 2002. CGMCC NO.0795 strains can be replaced by other M.Smegmatis strains.

The cells of mentioned vaccines can be crashed and split through various physical techniques. In our case, the high-pressure airflow shearing technique or high-pressure homogenization technique was employed.

The above mentioned M.S vaccine, aqueous vaccine or lyophilized preparations, consists of cellwall and tropina of Mycobacterium smegmatis as well as abundant immunologically competent CpG fragment compounds in bacteria DNA.

The mentioned M.S vaccine can be prepared as follows:
1) Culture of Thalli: The strain preserved at low temperature is dissolved at room temperature and seeded in Lowenstein-Jensen (modified) medium cultivated at 370 C for 3 to 7 days and subcultivated to Lowenstein-Jensen(modified) medium at 370 C for another 3 to 5 days.
2) Collection of Thalli: When they grow to log phase, the thalli on the surface of the culture media will be washed down by 0.9% sodium chloride solution, then washed again by 0.9% sodium chloride solution and collected after centrifuging at 40 C at the rate of 6000 r/min for 30 minutes.
3) Preparation of Vaccine: The collected thalli will be diluted into required concentration by adding 0.9% sodium chloride solution or a phosphate buffer solution, by being further split physically and sterilized.
4) Preparation of Final Product: The above solution will be packed as liquid M.S vaccines, or made into lyophilized preparations after lyophillization.

Now, it should be pointed out that the 2000 ml modified Lowenstein-Jensen medium is made according to the following procedures: 4.5 g L-Asparagine, 3.0 g potassium dihydrogen phosphate, 0.3 g MgSO4, 0.65 g magnesia lemonade, 37.6 g potato starch, 15.0 ml Glycerol and 750 ml distilled water are mixed, heated to boiling, and autoclaved for 20 minutes at 1210 C to get substratum. Fresh eggs are cleaned and dried, soaked in Alcohol of 75% for 30 minutes, taken out, put into a sterile room to dry. Then, the eggs are cracked and in total 1250 ml of egg liquid is put into a vessel, mixed completely and filtered through sterile gauze into a large triangle flask containing the previously prepared substratum. 20 ml malachite green of 2% is also put into the flask, mixed well and poured into a wide-mouthed bottle, and later put separately into middle-sized tubes, about 7 ml for each. Then the tubes are put in ovens for 40 minutes at 870 C-880 C. 24 hours later, a sterility test is done at 370 C and those passing the test are put in refrigerators for future use.

From the introduction of preparation of M.S vaccine, it is obvious that the M.S Vaccine is of some superior properties.

Generally speaking, this new vaccine has the following good points. Firstly, this vaccine makes good use of the adjuvant function of the cellwall, the antigenic specificity of the tropina which can also stimulate the production of cytokines and is abundant in immunologically competent CpG segments of bacteria DNA. Secondly, this vaccine is safer. At home and abroad, vaccines of the same kind are often of larger thalli and aqueous vaccines can easily be formed into cenobia, which may lead to notable side effects. The new vaccine can successfully overcome such problems by decreasing or even avoiding side effects. Thirdly, it is of high efficacy. This new vaccine can be injected many times. So it is more effective than vaccines of the same kind which can only be injected intradermally once or be injected for several times with the interval of more than three months for the notable side effects. Fourthly, such vaccine can not only provide prevention for the normal population, but can also prevent MTB carriers from being attacked by tuberculosis effectively and economically. Therefore, this vaccine is of great significance in the control of TB and in promoting quality of life. The last but not the least point, the vaccine is of therapeutic value for dermatosis mediated by the immune system or diseases characterized by higher Th2 immune responses.

### DESCRIPTION OF DRAWINGS

FIG. 1 is a diagram showing the appearance of organs of control animals.

FIG. 2 is a diagram showing the appearance of organs in treatment group.

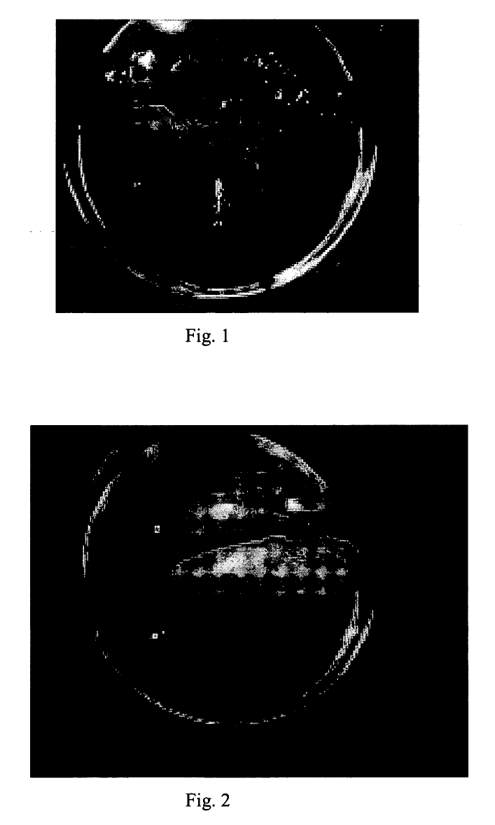

### DETAILED PROCEDURES FOR PREPARING THE M.S VACCINE

Now, the detailed description of the invention should be made companied with the figures and examples. But the following examples are offered by way of illustration and are not limiting.

### EXAMPLE 1

### Preparation of the M.S Vaccine

1) Culture of Thalli: The strain preserved at low temperature is dissolved at room temperature and seeded in Lowenstein-Jensen (modified) medium cultivated at 370 C for 3 to 7 days and subcultivated to Lowenstein-Jensen(modified) medium at 370 C for another 3 to 5 days.

2) Collection of Thalli: When they grow to log phase, the thalli on the surface of the culture media will be washed down by 0.9% sodium chloride solution, washed again by 0.9% sodium chloride solution and collected after centrifuging at 40 C at the rate of 6000 r/min for 30 minutes.

3) Preparation of Vaccine: The collected thalli will be diluted into required concentration by adding 0.9% sodium chloride solution or a phosphate buffer solution, by being further split physically and sterilized.

4) Preparation of Final Product: The above solution will be packed as liquid M.S vaccines, or made into lyophilized preparations after lyophillization.

Now, it should be pointed out that the 2000 ml Lowenstein-Jensen (modified )medium is made according to the following procedures: 4.5 g L-Asparagine, 3.0 g potassium dihydrogen phosphate, 0.3 g MgSO4, 0.65 g magnesia lemonade, 37.6 g potato starch, 15.0 ml glycerol and 750 ml distilled water are mixed, heated to boiling, and autoclaved for 20 minutes at 1210 C to get substratum. Fresh eggs are cleaned and dried, soaked in Alcohol of 75% for 30 minutes, taken out, put into a sterile room to dry. Then, the eggs are cracked and in total 1250 ml of egg liquid is put into a vessel, mixed completely and filtered through sterile gauze into a large triangle flask containing the previously prepared substratum. 20 ml malachite green of 2% is also put into the flask, mixed well and poured into a wide-mouthed bottle, and later put separately into middle-sized tubes, about 7 ml for each. Then the tubes are put in ovens for 40 minutes at 870 C-880 C. 24 hours later, a sterility test is done at 370 C and those passing the test are put in refrigerators for future use.

Animal Experiments

### Example 2

### Experiments on Guinea-Pigs Infected with TB

Normal guinea-pigs were injected subcutaneously with powerful TB strains. Three days later, the treatment group were injected intramuscularly with M.S vaccines, and once a week for four weeks, while the control group were injected with 0.9% sodium chloride solution.

Seven weeks after infection, all the animals were dissected and the pathological changes of various organs such as the liver, spleen, lungs and lymph nodes were observed and scored by the double blind method according to The Evaluation Criterion of Pathological Changes in Patients infected with TB in the book Experimental Methods in Pathology. Furthermore, pathological tests were made on the liver, spleen and lungs of the animals, and the results are listed in Table 1, while the appearance of those organs is shown in Photographs 1 and 2. In addition, all of the spleens were homogenized and assayed for colony forming units (CFU) of M. tuberculosis and the logarithm of the number of M.TB is shown in Table 2.

TABLE 1
Pathological Changes in Various Organs (X ± SD)

| | Negative | M.S Low-dose | M.S High-dose |
|---|---|---|---|
| | Control Group | Group | Group |
| Pathological | 64.6 ± 21.0 | 30.0 ± 15.3** | 27.5 ± 12.0** |
| Index | | | |

**When the treatment group and control group are compared, p < 0.01.

TABLE 2
Logarithm of the Number of M.TB Separated from spleens (X ±
SD)

| | | M.S | |
|---|---|---|---|
| | Negative Control | Low-dose | M.S High-dose |
| | Group | Group | Group |
| Logarithm | 4.69 ± 0.67 | 3.59 ± 1.66 | 3.98 ± 0.65* |
| of Viable | | | |
| Organisms | | | |
| from Spleen | | | |

*When the treatment group and control group are compared, p < 0.05.

Pathological sections show that there are widely spread grain-like tubercles or caseous necrosis in the lungs, liver and spleen of control animals, while only slight pathological changes occur to organs of animals in the treatment group as there only appear non-specific hyperplasia, post-treatment epithelium-like tubercles or even normal histological sections.

It can be concluded from the above results that by injecting M.S vaccines to guinea-pigs infected with M.TB pathological changes of various organs can be dramatically mitigated, pathological index can be cut down and M.TB can be depressed and even killed. Therefore, M.S vaccines are of preventive or therapeutic effect to guinea-pigs infected with TB.

### Example 3

### Pharmacological Study of M.S Vaccines

The immunological regulation and mechanism of M.S vaccines was studied by injecting the vaccines to the normal animal group, the group of inadequate immune function and that of hypersensitive. It is found that by injecting M.S vaccines the immune function of normal animals, especially Th1 immunological responses, consisting of T lymphproliferation responses and delayed type hypersensitivity are strengthened, and the secretion of Th1 cytokines such as IL-12,IFN-γ and IL-2 as well as killers in macrophage such as NO are elevated, which is of great significance to the prevention of intracellular infectious diseases like TB. Furthermore, by injecting M.S vaccines, the hypofunction of immune system can be promoted while the hyperfunction can be depressed. Therefore, M.S vaccines are of a two-way immune regulatory function and can provide prevention for people at different M.TB infectious phase.

### Example 4

### Toxicological Study of M.S Vaccine

According to new drugs' preclinical study requirements, M.S vaccine's acute toxicity testing, chronical toxicity testing, systematic anaphylaxis experiment and heat source experiment were made on mice, Beagle's dogs, guinea-pigs and rabbits respectively. 25000, 50000 and 250000 times of human dosage were given to mice by intraperitoneal or intramuscular injection, and no abnormal symptoms and death follow. Beagle's dogs were given intramuscular injection for 14 weeks successively with 62.5, 625 and 3125 times of human dosage and no toxic reaction was found by checking general behavior, blood routine, blood biochemistry, urine routine, ECG and pathological indexes at the early, medium, later and convalescent stage of the experiment. Besides, no heat reaction and systematic immediate allergy was caused by M.S vaccine in heat source experiment and systematic allergic test.

In short, it is shown by experiments that the M.S vaccine can strengthen the normal cellular immune function, promote the recovery of normal immune function in mice with hypofunction and suppress the immunogical response in guinea-pigs with hyperfunction. In the experiment animals infected with tubercle bacillus, the multiplication of tubercle bacillus was suppressed significantly and pathological changes of various organs mitigated in turn. Animal allergic reaction was also suppressed significantly in hog blood serum allergic experiment. Besides, this vaccine is of good safety.

It should be pointed out that any minor modifications and substitute with nothing new in the precept should be regarded as infringing the patent.

We claim:

1. A mycobacterium smegmatis vaccine, characterized by: it is a split product of Mycobacterium smegmatis CGMCC NO.0795.

2. The mycobacterium smegmatis vaccine according to claim 1, characterized by: said vaccine is prepared by splitting cells by a physical method.

3. The mycobacterium smegmatis vaccine according to claim 1, characterized by: said vaccine consists of cellwall and tropina of Mycobacterium smegmatis as well as abundant immunologically competent CpG fragment compounds of bacteria DNA.

4. The mycobacterium smegmatis vaccine according to claim 3, characterized by: said compound is aqueous vaccine or lyophilized preparations.

5. The method of vaccine preparation, characterized by: the vaccine according to any one of above 4 claims is made from Mycobacterium smegmatis.

6. The use of vaccine according to any one of above 5 claims in a biological product preparation, characterized by: said biological product can be used for prevention and as treatment to MTB carriers; This biological product can also be employed to promote the recovery of normal immune function in TB patients with hypofunction, and depress the immunological responses in TB patients with hyperfunction.

7. The use according to claim 6, characterized by: said biological product can be used for the elevation of Th1 immunological response in the normal population.
